(19) United States
(12) Patent Application Publication
(10) Pub. No.: US 2025/0318845 A1
(43) Pub. Date: Oct. 16, 2025

(54) MEDICAL DEVICES FOR USE DURING A STONE REMOVAL INTERVENTION

(71) Applicant: Boston Scientific Scimed, Inc., Maple Grove, MN (US)

(72) Inventors: Eve George, Watertown, MA (US); Jeffrey A. Meganck, North Grafton, MA (US); Cristian Parisi, Amherst, MA (US); Yen-Hao Hsu, Shrewsbury, MA (US)

(73) Assignee: Boston Scientific Scimed, Inc., Maple Grove, MN (US)

(21) Appl. No.: 19/176,439

(22) Filed: Apr. 11, 2025

Related U.S. Application Data
(60) Provisional application No. 63/633,203, filed on Apr. 12, 2024.

(51) Int. Cl. A61B 17/22; A61B 18/00; A61B 18/26; A61M 5/158; A61M 31/00

(52) U.S. Cl. CPC A61B 17/2202; A61B 18/26; A61M 31/00; A61B 2017/22025; A61B 2017/22038; A61B 2017/22082; A61B 2018/00511; A61B 2018/00982; A61M 5/158; A61M 2210/1082

(57) ABSTRACT

Medical devices for use during a stone removal intervention as well as methods for treating a stone along a urinary tract are disclosed. An example medical device may include an elongate shaft having an injection lumen formed therein. The elongate shaft may be configured to navigate through a body lumen or a body tissue to a position adjacent to a stone disposed along a urinary tract and/or adjacent to a kidney. The capturing substance may be disposed within the injection lumen and may be configured to be injected into the urinary tract adjacent to the stone.

### CROSS-REFERENCE TO RELATED APPLICATIONS

This application claims the benefit of U.S. Provisional Patent Application Ser. No. 63/633,203 filed on Apr. 12, 2024, the disclosure of which is incorporated herein by reference.

### TECHNICAL FIELD

The present disclosure pertains to medical devices, and methods for manufacturing and using medical devices. More particularly, the present disclosure pertains to medical devices for use during a stone removal intervention.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A medical device for use during a stone removal intervention is disclosed. The medical device comprises: an elongate shaft having an injection lumen formed therein; wherein the elongate shaft is configured to navigate through a body lumen or a body tissue to a position adjacent to a stone disposed along a urinary tract and/or adjacent to a kidney; and a capturing substance disposed within the injection lumen and configured to be injected into the urinary tract adjacent to the stone.

Alternatively or additionally to any of the embodiments above, the capturing substance includes a hydrogel.

Alternatively or additionally to any of the embodiments above, the capturing substance includes a hydrophilic polymer.

Alternatively or additionally to any of the embodiments above, the capturing substance includes polyethylene glycol, polyoxazoline, polyvinylpyrrolidone, poly(2-hydroxyethyl methacrylate), or combinations thereof.

Alternatively or additionally to any of the embodiments above, the capturing substance includes a shear-thinning material.

Alternatively or additionally to any of the embodiments above, the capturing substance includes a conformable material.

Alternatively or additionally to any of the embodiments above, the capturing substance is configured to form a barrier within the urinary tract.

Alternatively or additionally to any of the embodiments above, the capturing substance is configured to capture one or more stone fragments.

Alternatively or additionally to any of the embodiments above, the elongate shaft is configured to navigate beyond the stone by passing the elongate shaft over a guidewire.

Alternatively or additionally to any of the embodiments above, further comprising a ureteroscope, wherein the elongate shaft is configured to extend through the ureteroscope.

Alternatively or additionally to any of the embodiments above, the elongate shaft comprises a needle.

A method for treating a stone along a urinary tract is disclosed. The method comprises: advancing an injection sheath along the urinary tract and/or through a body tissue to a position adjacent to the stone; injecting a capturing substance into the urinary tract through the injection sheath; performing an intervention on the stone that causes the stone to break into a plurality of fragments; and capturing at least some of the plurality of fragments with the capturing substance.

Alternatively or additionally to any of the embodiments above, advancing an injection sheath along the urinary tract and/or through a body tissue to a position adjacent to the stone includes advancing the injection sheath over a guidewire.

Alternatively or additionally to any of the embodiments above, advancing an injection sheath along the urinary tract and/or through a body tissue to a position adjacent to the stone includes advancing the injection sheath through a ureteroscope.

Alternatively or additionally to any of the embodiments above, the injection sheath includes a needle and wherein advancing an injection sheath along the urinary tract and/or through a body tissue to a position adjacent to the stone includes advancing the needle to a location adjacent to the stone.

Alternatively or additionally to any of the embodiments above, the capturing substance includes a hydrogel, and wherein injecting a capturing substance into the urinary tract through the injection sheath includes injecting the hydrogel into the urinary tract.

Alternatively or additionally to any of the embodiments above, the capturing substance includes a shear-thinning material, and wherein injecting a capturing substance into the urinary tract through the injection sheath includes injecting the shear-thinning material into the urinary tract.

Alternatively or additionally to any of the embodiments above, injecting a capturing substance into the urinary tract through the injection sheath includes injecting a plurality of doses of the capturing substance into the urinary tract.

Alternatively or additionally to any of the embodiments above, performing an intervention on the stone that causes the stone to break into a plurality of fragments includes lithotripsy.

A medical device for use as part of a lithotripsy procedure is disclosed. The medical device comprises: an injection sheath configured to navigate to a position adjacent to a stone disposed along a urinary tract and/or adjacent to a kidney; a capturing substance configured to be injected into the urinary tract adjacent to the stone through the injection sheath; and wherein the capturing substance includes a hydrogel, a shear-thinning material, a hydrophilic polymer, or a combination thereof.

Alternatively or additionally to any of the embodiments above, the injection sheath includes an elongate shaft configured to be advanced over a guidewire.

Alternatively or additionally to any of the embodiments above, the injection sheath includes an elongate shaft configured to be advanced through a ureteroscope.

Alternatively or additionally to any of the embodiments above, the injection sheath includes a needle.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically depicts a stone disposed along a urinary tract.

FIG. 2 illustrates an example medical device disposed adjacent to a stone disposed along a urinary tract.

FIG. 3 illustrates the injection of a capturing substance along the urinary tract adjacent to a stone.

FIG. 4 schematically depicts the use of the capturing substance along the urinary tract.

FIG. 5 illustrates an example medical device disposed adjacent to a stone disposed along a urinary tract.

FIG. 6 schematically depicts a stone disposed adjacent to a kidney.

FIG. 7 illustrates the injection of a capturing substance adjacent to a stone.

FIG. 8 illustrates the injection of a capturing substance adjacent to a stone.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Obstructions such as kidney stones along the urinary tract and/or adjacent the kidney are a common problem among patients. A number of interventions have been developed for treating kidney stones such as stone retrieval devices, ureteroscopy, percutaneous nephrolithotomy (PCNL), lithotripsy, etc. Some of these interventions, for example lithotripsy, include the breaking up of the stones into a number of smaller fragments, which may be easier for a patient to pass. It may be desirable to minimize the possibility that stone fragments can migrate (e.g., migrate upstream) along the urinary tract. In addition, it may be desirable to capture and/or encapsulate at least some of the stone fragments. Disclosed herein are devices that can be used in conjunction with an intervention aimed at treating/removing obstructions/stones along the urinary tract and/or adjacent the kidney. Some additional details regarding these devices are disclosed herein.

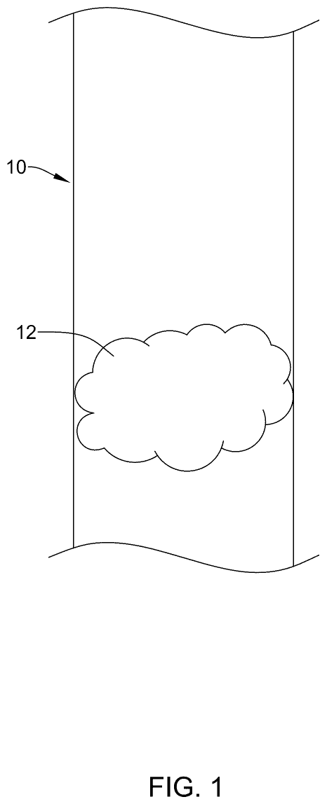

FIG. 1 schematically depicts a section of a urinary tract, for example a portion of the ureter 10, having an obstruction or stone 12 disposed therein. It can be appreciated that FIG. 1 is schematic in nature and can be understood to represent any suitable portion of the urinary tract including the kidneys and/or adjacent to the kidneys. The stone 12 may represent a kidney stone or any other suitable obstruction. It may be desirable to treat/remove the stone 12, for example using an intervention. One example intervention that may be used to treat/remove the stone 12 may include lithotripsy, where a sonic wave and/or shock wave is directed at the stone 12 in order to break the stone 12 into smaller fragments. Other interventions are contemplated including interventions where a laser is directed at the stone 12 in order to break the stone 12 into smaller fragments.

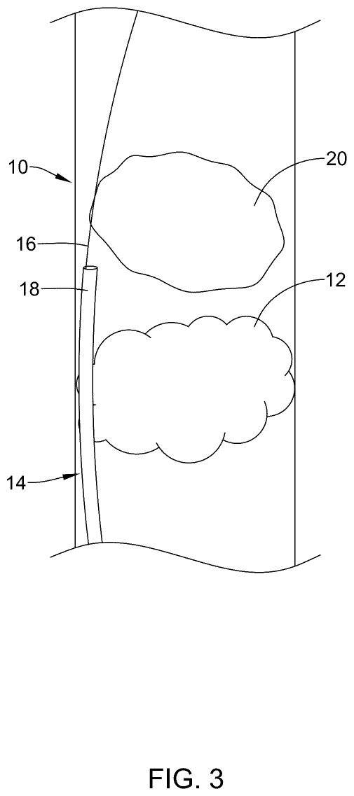

In at least some instances, it may be desirable to reduce the likelihood that the stone fragments may migrate (e.g., migrate upstream) within the urinary tract. For example, energy delivered by an intervention that may break the stone 12 into smaller fragments may cause fragments to migrate and/or otherwise be propelled upstream. Furthermore, the stone fragments may have relatively sharp surfaces or edges that could result in patient discomfort as the fragments are advanced/passed. In at least some instances, a capturing substance (e.g., the capturing substance 20, first depicted in FIG. 3) can be injected and/or delivered adjacent to the stone 12. The capturing substance can provide a number of desirable benefits. For example, the capturing substance can form a barrier that can help to reduce the likelihood that stone fragments can migrate within the urinary tract. Furthermore, the capturing substance can help to catch or capture at least some of the stone fragments, thereby helping to make it easier for a patient to pass the stone fragments. For example, at least some of the stone fragments may be captured within the capturing substance. This may effectively encapsulate the stone fragments within the capturing substance, thereby helping to smooth some of the edge surfaces of the stone fragments that may otherwise be relatively sharp, which may make it easier for a patient to pass the stone fragments.

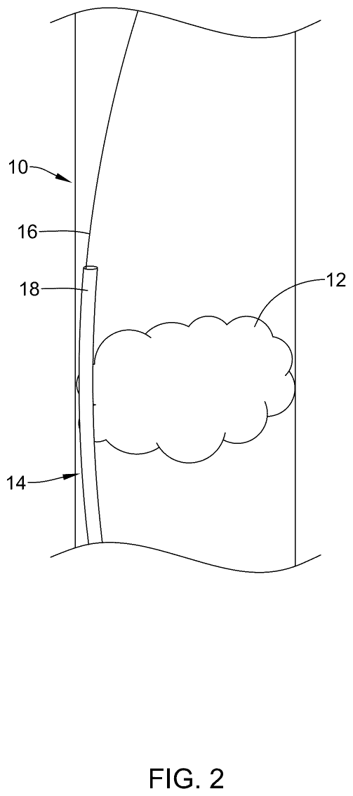

FIG. 2 illustrates a delivery system 14 that may be used in conjunction with a stone removal intervention or similar procedure, for example to deliver the capturing substance. In this example, the system 14 may include a guidewire 16 and an elongate sheath or injection sheath 18 slidable along the guidewire 16. In use, the guidewire 16 may be navigated through the ureter 10 to a position past (e.g., upstream) of the stone 12. The injection sheath 18 may be advanced over the guidewire 16 so that the distal end of the injection sheath 18 is disposed past (e.g., upstream) of the stone 12. The injection sheath 18 may be used to inject a capturing substance 20 into the ureter at a position upstream of the stone 12 as depicted in FIG. 3. For example, the injection sheath 18 may have an injection lumen formed therein that allows a clinician to inject the capturing substance 20 adjacent to the stone 12. The structure of the injection sheath 18 (and/or the injection mechanism, which may include a syringe) can vary. For example, the injection sheath 18 may include a tubular structure connectable to a delivery device such as a syringe. Other structures/arrangements are contemplated.

In at least some instances the capturing substance 20 may take the form of a hydrogel. The hydrogel capturing substance 20 may be desirable for a number of reasons. Hydrogels may be biocompatible, may be absorbed by the patient, may be relatively easy for the patient to pass, and/or have other benefits. Other suitable materials may include hydrophilic polymers, polyethylene glycol, polyoxazoline, polyvinylpyrrolidone, poly(2-hydroxyethyl methacrylate), combinations thereof, and/or the like. In some instances, the capturing substance 20 may include a conformable material and/or shear thinning material or composition and/or a gelatin-based shear-thinning material (e.g., a gelatin-based shear-thinning hydrogel). The shear thinning material, for example, may include a gelatin or gelatin derivative and silicate nanoparticles. In some instances, the shear thinning material may also include a radiopaque material. For example, the capturing substance 20 may include OBSIDIO™ Conformable Embolic material, commercially available from Boston Scientific.

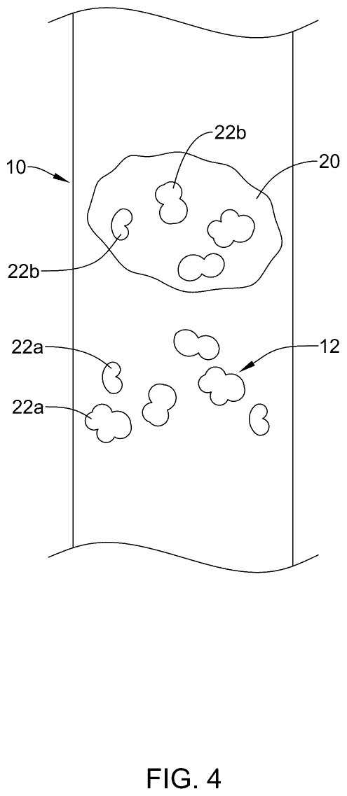

In at least some instances, after injecting the capturing substance 20 along the urinary tract and/or adjacent to a kidney, an intervention such as lithotripsy may be used to break the stone 12 into smaller fragments. For example, FIG. 4 illustrates that the stone 12, after a lithotripsy procedure, may be broken into fragments such as fragments 22a, 22b. It is noted that for the purposes of brevity, the lithotripsy procedure is not depicted in the drawings. However, it can be appreciated that a number of different lithotripsy procedures and/or lithotripsy devices may be used to break the stone 12 into fragments 22a, 22b.

The capturing substance 20 may help to prevent fragments 22a of the stone 12 from migrating upstream within the ureter 10. This may include the capturing substance 20 functioning as a barrier that can substantially prevent the fragments 22a from migrating. In addition, the capturing substance 20 may also capture fragments 22b of the stone 12. This may also help to prevent the fragments 22b from migrating and/or help to cover/smooth edges (e.g., sharp edges) of the fragments 22b. Because of this, the capturing substance 20 may help to cushion fragments 22b so that the fragments 22b can be passed more easily.

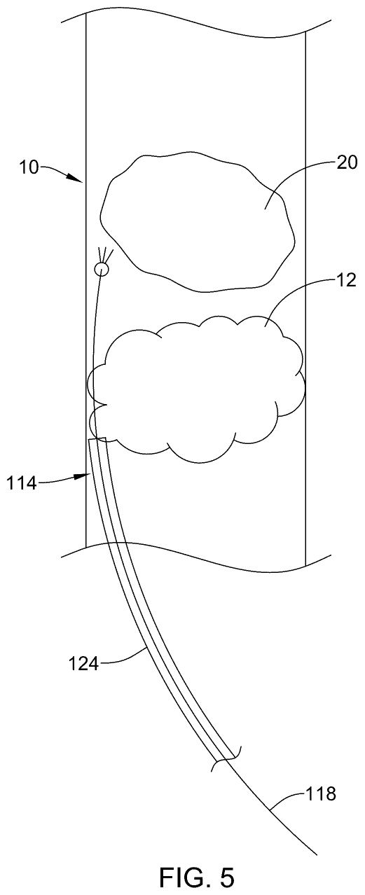

While FIGS. 2-4 illustrate that the injection sheath 18 can be used to deliver the capturing substance 20, this is not intended to be limiting as different delivery systems/mechanisms may be utilized, depending on the intervention. For example, FIG. 5 schematically illustrates another example delivery system 114. The delivery system 114 may use a scope device such as a ureteroscope 128. An injection device or sheath 118 may be advanced through the ureteroscope 128 past the stone 12. The injection sheath 118 may be used to deliver/inject the capturing substance 20. The capturing substance 20 may function similarly to what is described herein.

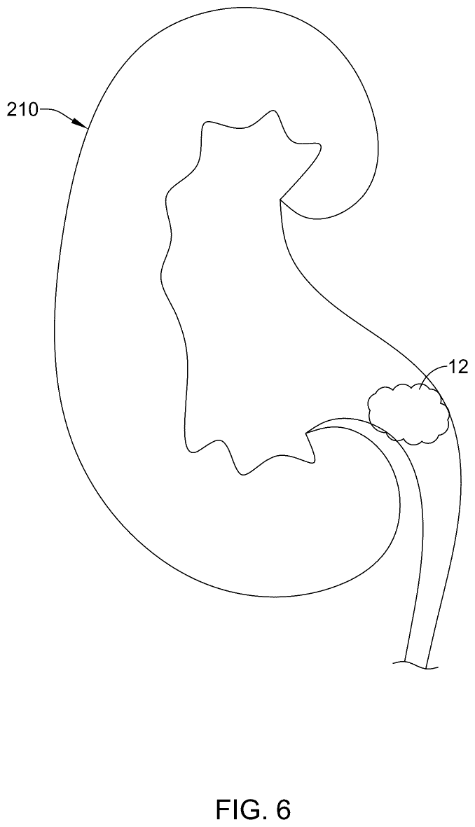

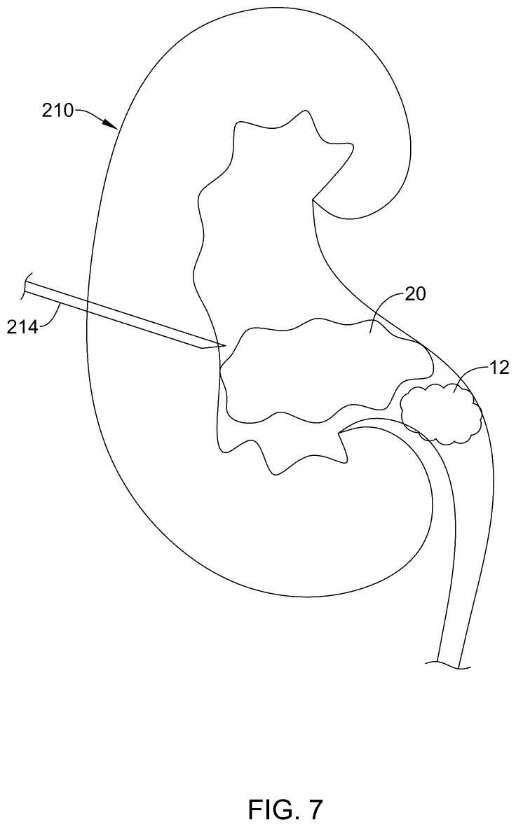

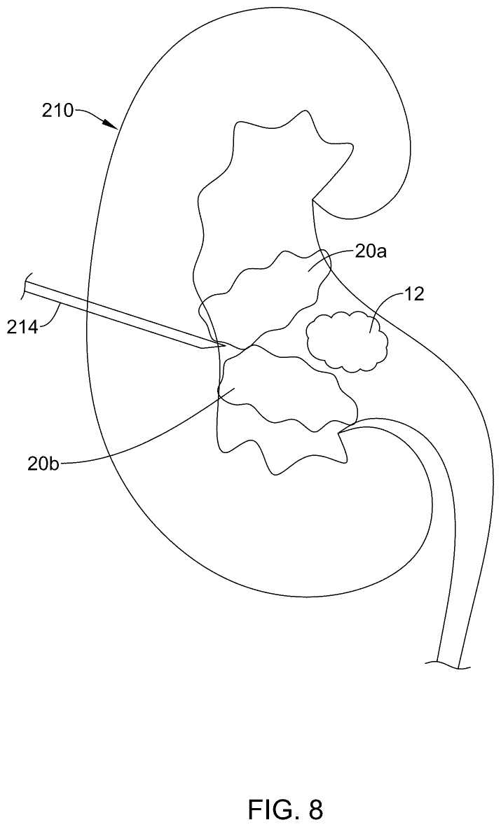

FIG. 6 illustrates that in some instances, the stone 12 may be disposed adjacent to a kidney 210. For example, the stone 12 may disposed adjacent to a renal pelvis. It may be challenging to access the stone 12, particularly at a location upstream of the stone 12, using a catheter/sheath/scope system. In such instances, it may be desirable to use a needle to access the kidney 210. For example, FIG. 7 depicts a delivery system 214, which may take the form of a needle. The delivery system 214 may be used to deliver/inject the capturing substance 20. If desired, the delivery system 214 may be used to deliver a plurality of capturing substances 20a, 20b as depicted in FIG. 8.

The materials that can be used for the various components of the systems 14/114 (and/or other systems disclosed herein) may include those commonly associated with medical devices. For example, the systems 14/114, and/or the components thereof, may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device for use during a stone removal intervention, the medical device comprising:
an elongate shaft having an injection lumen formed therein;
wherein the elongate shaft is configured to navigate through a body lumen or a body tissue to a position adjacent to a stone disposed along a urinary tract and/or adjacent to a kidney; and
a capturing substance disposed within the injection lumen and configured to be injected into the urinary tract adjacent to the stone.

2. The medical device of claim 1, wherein the capturing substance includes a hydrogel.

3. The medical device of claim 1, wherein the capturing substance includes a hydrophilic polymer.

4. The medical device of claim 1, wherein the capturing substance includes polyethylene glycol, polyoxazoline, polyvinylpyrrolidone, poly(2-hydroxyethyl methacrylate), or combinations thereof.

5. The medical device of claim 1, wherein the capturing substance includes a shear-thinning material.

6. The medical device of claim 1, wherein the capturing substance includes a conformable material.

7. The medical device of claim 1, wherein the capturing substance is configured to form a barrier within the urinary tract.

8. The medical device of claim 1, wherein the capturing substance is configured to capture one or more stone fragments.

9. The medical device of claim 1, wherein the elongate shaft is configured to navigate beyond the stone by passing the elongate shaft over a guidewire.

10. The medical device of claim 1, further comprising a ureteroscope, wherein the elongate shaft is configured to extend through the ureteroscope.

11. The medical device of claim 1, wherein the elongate shaft comprises a needle.

12. A method for treating a stone along a urinary tract, the method comprising:
advancing an injection sheath along the urinary tract and/or through a body tissue to a position adjacent to the stone;
injecting a capturing substance into the urinary tract through the injection sheath;
performing an intervention on the stone that causes the stone to break into a plurality of fragments; and
capturing at least some of the plurality of fragments with the capturing substance.

13. The method of claim 12, wherein advancing an injection sheath along the urinary tract and/or through a body tissue to a position adjacent to the stone includes advancing the injection sheath over a guidewire.

14. The method of claim 12, wherein advancing an injection sheath along the urinary tract and/or through a body tissue to a position adjacent to the stone includes advancing the injection sheath through a ureteroscope.

15. The method of claim 12, wherein the injection sheath includes a needle and wherein advancing an injection sheath along the urinary tract and/or through a body tissue to a position adjacent to the stone includes advancing the needle to a location adjacent to the stone.

16. The method of claim 12, wherein the capturing substance includes a hydrogel, and wherein injecting a capturing substance into the urinary tract through the injection sheath includes injecting the hydrogel into the urinary tract.

17. The method of claim 12, wherein the capturing substance includes a shear-thinning material, and wherein injecting a capturing substance into the urinary tract through the injection sheath includes injecting the shear-thinning material into the urinary tract.

18. The method of claim 12, wherein injecting a capturing substance into the urinary tract through the injection sheath includes injecting a plurality of doses of the capturing substance into the urinary tract.

19. The method of claim 12, wherein performing an intervention on the stone that causes the stone to break into a plurality of fragments includes lithotripsy.

20. A medical device for use as part of a lithotripsy procedure, the medical device comprising:
an injection sheath configured to navigate to a position adjacent to a stone disposed along a urinary tract and/or adjacent to a kidney;
a capturing substance configured to be injected into the urinary tract adjacent to the stone through the injection sheath; and
wherein the capturing substance includes a hydrogel, a shear-thinning material, a hydrophilic polymer, or a combination thereof.
